(19) United States
(12) Patent Application Publication
(10) Pub. No.: US 2021/0259866 A1
(43) Pub. Date: Aug. 26, 2021

(54) TAPERED MULTILAYER CORE MEMBER FOR MEDICAL DEVICE DELIVERY SYSTEMS

(71) Applicant: Covidien LP, Mansfield, MA (US)

(72) Inventors: Ashok Nageswaran, Irvine, CA (US); James Davidson, San Juan Capistrano, CA (US)

(21) Appl. No.: 16/802,227

(22) Filed: Feb. 26, 2020

(51) Int. Cl. A61F 2/966; A61L 29/02

(52) U.S. Cl. CPC A61F 2/966; A61L 29/02; A61F 2210/0014; A61F 2240/001; A61F 2250/0036

(57) ABSTRACT

A core member and associated systems and methods are disclosed herein. In some embodiments, the core member comprises a first portion including first and second materials, and a second portion distal to the first portion and including only the first material. The first and second portions can each be tapered in a continuous or discontinuous manner. The second portion can have a minimum length that is substantially straight and heat-treated or aged to have a minimum strength modulus.

### TECHNICAL FIELD

The present technology relates to core members for use with medical devices and, in particular embodiments, to tapered multilayer core members for use with medical devices.

### BACKGROUND

Walls of the vasculature, particularly arterial walls, may develop areas of pathological dilatation called aneurysms that often have thin, weak walls that are prone to rupturing. Aneurysms are generally caused by weakening of the vessel wall due to disease, injury, or a congenital abnormality. Aneurysms occur in different parts of the body, and the most common are abdominal aortic aneurysms and cerebral (e.g., brain) aneurysms in the neurovasculature. When the weakened wall of an aneurysm ruptures, it can result in death, especially if it is a cerebral aneurysm that ruptures.

Aneurysms are generally treated by excluding or at least partially isolating the weakened part of the vessel from the arterial circulation. For example, conventional aneurysm treatments include: (i) surgical clipping, where a metal clip is secured around the base of the aneurysm; (ii) packing the aneurysm with small, flexible wire coils (micro-coils); (iii) using embolic materials to “fill” an aneurysm; (iv) using detachable balloons or coils to occlude the parent vessel that supplies the aneurysm; and (v) intravascular stenting.

Intravascular stents are well known in the medical arts for the treatment of vascular stenoses or aneurysms. Stents are prostheses that expand radially or otherwise within a vessel or lumen to support the vessel from collapsing. Methods for delivering these intravascular stents are also well known. Conventional methods of introducing a compressed stent into a vessel and positioning it within an area of stenosis or an aneurysm often include percutaneously advancing a distal portion of a guiding catheter through the vascular system of a patient until the distal portion is proximate the stenosis or aneurysm. A second, inner catheter and a guidewire within the inner catheter are advanced through the distal portion of the guiding catheter. The guidewire, which carries the stent, is then advanced out of the distal portion of the guiding catheter into the vessel until the distal portion of the guidewire and the stent are positioned at the point of the lesion within the vessel.

### SUMMARY

The subject technology is illustrated, for example, according to various aspects described below, including with reference to FIGS. 1-6B. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., Clause 1, Clause 28, Clause 55, etc. The other clauses can be presented in a similar manner

Clause 1. A core member for use with a medical device, comprising:
a first portion comprising a first material and a second material surrounding the first material along a length of the first portion, the first material extending along substantially an entire length of the core member, and
a second portion distal to the first portion and comprising only the first material, the second portion being tapered in a distal direction such that an outermost cross-sectional dimension at a proximal end of the second portion is greater than an outermost cross-sectional dimension at a distal end of the second portion, the second portion having a minimum length.

Clause 2. The core member of Clause 1, wherein the minimum length is at least 0.5 inches, 1.0 inches, 1.5 inches, 2.0 inches, 2.5 inches, 3.0 inches, 3.5 inches, 4.0 inches, 4.5 inches, 5.0 inches, 5.5 inches, or 6.0 inches.

Clause 3. The core member of Clause 2, wherein the second portion is substantially straight and/or does not exhibit curling.

Clause 4. The core member of any one of the preceding Clauses, wherein a distalmost section of the second portion is substantially straight and/or does not exhibit curling.

Clause 5. The core member of any one of the preceding Clauses, wherein the outermost cross-sectional at the distal end of the second portion is no more than 0.0015 inches, 0.0020 inches, 0.0025 inches, 0.0030 inches, 0.0035 inches, 0.0040 inches, 0.0045 inches, 0.0050 inches, 0.0055 inches, 0.0060 inches, 0.0065 inches, or 0.0070 inches.

Clause 6. The core member of any one of the preceding Clauses, wherein the outermost cross-sectional at the proximal end of the second portion is no more than 0.0025 inches, 0.0030 inches, 0.0035 inches, 0.0040 inches, 0.0045 inches, 0.0050 inches, 0.0055 inches, 0.0060 inches, 0.0065 inches, 0.0070 inches, 0.0075 inches, 0.0080 inches, or 0.0085 inches.

Clause 7. The core member of any one of the preceding Clauses, wherein the outermost cross-sectional of the first material at the first portion is at least 0.0050 inches, 0.0055 inches, 0.0060 inches, 0.0065 inches, 0.0070 inches, 0.0075 inches.

Clause 8. The core member of any one of the preceding Clauses, wherein the core member is ground such that the core member has a ground length of at least 20 inches, 25 inches, 30 inches, 35 inches, 40 inches, 45 inches, or 50 inches.

Clause 9. The core member of any one of the preceding Clauses, wherein the core member has an unground length of at least 30 inches, 35 inches, 40 inches, 45 inches, or 50 inches.

Clause 10. The core member of any one of the preceding Clauses, wherein the second portion is heat treated.

Clause 11. The core member of any one of the preceding Clauses, wherein discrete areas of the second portion are heat treated.

Clause 12. The core member of any one of the preceding Clauses, wherein a modulus of the core member measured at the distal end of the second portion is at least 60 gigapascals (GPa), 65 GPa, 70 GPa, 75 GPa, 80 GPa, 85 GPa, 90 GPa, 95 GPa, or 100 GPa.

Clause 13. The core member of any one of the preceding Clauses, wherein the modulus of second portion decreases in a distal direction.

Clause 14. The core member of any one of the preceding Clauses, wherein the second portion includes a first area having a first modulus and a second area having a second modulus greater than the first modulus, the second area being distal and tapered relative to the first area.

Clause 15. The core member of any one of the preceding Clauses, wherein the first material comprises Nitinol, titanium, stainless steel, chromium, cobalt, and/or alloys thereof.

Clause 16. The core member of any one of the preceding Clauses, wherein the first material comprises titanium beta III.

Clause 17. The core member of any one of the preceding Clauses, wherein the second material comprises Nitinol, titanium, stainless steel, chromium, cobalt, and/or alloys thereof.

Clause 18. The core member of any one of the preceding Clauses, wherein the second material comprises a cobalt-chromium alloy.

Clause 19. The core member of any one of the preceding Clauses, wherein the second material comprises 35N LT.

Clause 20. The core member of any one of the preceding Clauses, wherein the core member comprises drawn filled tubing (DFT) wire.

Clause 21. The core member of any one of the preceding Clauses, wherein the core member comprises a continuous and/or contiguous surface extending along the substantially entire length of the core member.

Clause 22. The core member of any one of the preceding Clauses, wherein the second portion continuously tapers in a distal direction along the length of the second portion.

Clause 23. The core member of any one of the preceding Clauses, wherein the second material in the first portion comprises a first thickness, the core member further comprising a third portion between the first and second portions, the third portion comprising the first material and the second material surrounding the first material, the second material in the third portion having a second thickness less than the first thickness.

Clause 24. The core member of Clause 23, wherein the third portion is tapered in a distal direction such that an outermost cross-sectional at a proximal end of the third portion is greater than an outermost dimension at a distal end of the third portion.

Clause 25. The core member of Clause 23 or Clause 24, wherein a thickness of the first material in the third portion is substantially the same as a thickness of the first material in the first portion.

Clause 26. The core member of any one of the preceding Clauses, wherein the core member is a tipless core member.

Clause 27. The core member of any one of the preceding Clauses, wherein the core member is a pushwire or guidewire.

Clause 28. A core member for use with a medical device, comprising:
a first material extending along an entire length of the core member, the first material being tapered in a distal direction such that a thickness of the first material at a proximal end of the core member is greater than a thickness of the first material at a distal end of the core member;
a second material surrounding the first material for at least a portion of the length of the core member, the second material being tapered in a distal direction such that a thickness of the second material at the proximal end is greater than a thickness of the second material at the distal end; and
   a distalmost section comprising (i) only the first material, (ii) an outermost cross-sectional dimension of no more than 0.070 inches, and (iii) a length of at least 0.5 inches, the distalmost section being substantially straight.

Clause 29. The core member of Clause 28, wherein the minimum length is at least 0.5 inches, 1.0 inches, 1.5 inches, 2.0 inches, 2.5 inches, 3.0 inches, 3.5 inches, 4.0 inches, 4.5 inches, 5.0 inches, 5.5 inches, or 6.0 inches.

Clause 30. The core member of any one of the preceding Clauses, wherein the second portion is substantially straight and/or does not exhibit curling.

Clause 31. The core member of any one of the preceding Clauses, wherein a distalmost section of the second portion is substantially straight and/or does not exhibit curling.

Clause 32. The core member of any one of the preceding Clauses, wherein the outermost cross-sectional at the distal end of the second portion is no more than 0.0015 inches, 0.0020 inches, 0.0025 inches, 0.0030 inches, 0.0035 inches, 0.0040 inches, 0.0045 inches, 0.0050 inches, 0.0055 inches, 0.0060 inches, 0.0065 inches, or 0.0070 inches.

Clause 33. The core member of any one of the preceding Clauses, wherein the outermost cross-sectional at the proximal end of the second portion is no more than 0.0025 inches, 0.0030 inches, 0.0035 inches, 0.0040 inches, 0.0045 inches, 0.0050 inches, 0.0055 inches, 0.0060 inches, 0.0065 inches, 0.0070 inches, 0.0075 inches, 0.0080 inches, or 0.0085 inches.

Clause 34. The core member of any one of the preceding Clauses, wherein the outermost cross-sectional of the first material at the first portion is at least 0.0050 inches, 0.0055 inches, 0.0060 inches, 0.0065 inches, 0.0070 inches, 0.0075 inches.

Clause 35. The core member of any one of the preceding Clauses, wherein the core member is ground such that the core member has a ground length of at least 20 inches, 25 inches, 30 inches, 35 inches, 40 inches, 45 inches, or 50 inches.

Clause 36. The core member of any one of the preceding Clauses, wherein the core member has an unground length of at least 30 inches, 35 inches, 40 inches, 45 inches, or 50 inches.

Clause 37. The core member of any one of the preceding Clauses, wherein the second portion is heat treated.

Clause 38. The core member of any one of the preceding Clauses, wherein discrete areas of the second portion are heat treated.

Clause 39. The core member of any one of the preceding Clauses, wherein a modulus of the core member measured at the distal end of the second portion is at least 60 gigapascals (GPa), 65 GPa, 70 GPa, 75 GPa, 80 GPa, 85 GPa, 90 GPa, 95 GPa, or 100 GPa.

Clause 40. The core member of any one of the preceding Clauses, wherein the modulus of second portion decreases in a distal direction.

Clause 41. The core member of any one of the preceding Clauses, wherein the second portion includes a first area having a first modulus and a second area having a second modulus greater than the first modulus, the second area being distal and tapered relative to the first area.

Clause 42. The core member of any one of the preceding Clauses, wherein the first material comprises Nitinol, titanium, stainless steel, chromium, cobalt, and/or alloys thereof.

Clause 43. The core member of any one of the preceding Clauses, wherein the first material comprises titanium beta III.

Clause 44. The core member of any one of the preceding Clauses, wherein the second material comprises Nitinol, titanium, stainless steel, chromium, cobalt, and/or alloys thereof.

Clause 45. The core member of any one of the preceding Clauses, wherein the second material comprises a cobalt-chromium alloy.

Clause 46. The core member of any one of the preceding Clauses, wherein the second material comprises 35N LT.

Clause 47. The core member of any one of the preceding Clauses, wherein the core member comprises drawn filled tubing (DFT) wire.

Clause 48. The core member of any one of the preceding Clauses, wherein the core member comprises a continuous and/or contiguous surface extending along the substantially entire length of the core member.

Clause 49. The core member of any one of the preceding Clauses, wherein the second portion continuously tapers in a distal direction along the length of the second portion.

Clause 50. The core member of any one of the preceding Clauses, wherein the second material in the first portion comprises a first thickness, the core member further comprising a third portion between the first and second portions, the third portion comprising the first material and the second material surrounding the first material, the second material in the third portion having a second thickness less than the first thickness.

Clause 51. The core member of Clause 50, wherein the third portion is tapered in a distal direction such that an outermost cross-sectional at a proximal end of the third portion is greater than an outermost dimension at a distal end of the third portion.

Clause 52. The core member of Clause 50 or Clause 51, wherein a thickness of the first material in the third portion is substantially the same as a thickness of the first material in the first portion.

Clause 53. The core member of any one of the preceding Clauses, wherein the core member is a tipless core member.

Clause 54. The core member of any one of the preceding Clauses, wherein the core member is a pushwire or guidewire.

Clause 55. A medical device delivery system, comprising:
a core assembly sized for insertion into a corporeal lumen, the core assembly comprising the core member of any one of the preceding clauses.

Clause 56. The medical device delivery system of Clause 55, further comprising an implant or stent disposed around the core member, the core member being configured to carry the implant or stent.

Clause 57. The medical device delivery system of any one of the preceding Clauses, further comprising a catheter including a lumen configured to receive the core assembly therethrough.

Clause 58. The medical device delivery system of any one of the preceding Clauses, further comprising a first catheter, and a second catheter disposed within the first catheter, the first catheter including a lumen configured to receive the core assembly therethrough.

Clause 59. The medical device delivery system of any one of the preceding Clauses, wherein the system does not include a hypotube.

Clause 60. A method of manufacturing a core member for use with a medical device, comprising:
providing a first elongate structure comprising a first material and a second material surrounding the first material, the first and second materials extending along a length of the first elongate structure; and
removing portions of the first elongate structure to form a second elongate structure comprising (i) a first portion including the first and second materials, and (ii) a second portion distal to the first portion and including only the first material, wherein—
   the first portion is tapered in a distal direction such that an outermost cross-sectional dimension at a proximal end of the first portion is greater than an outermost cross-sectional dimension at a distal end of the first portion,
   the second portion is tapered in a distal direction such that an outermost cross-sectional dimension at a proximal end of the second portion is greater than an outermost cross-sectional dimension at a distal end of the second portion, and
   the second portion has a minimum length.

Clause 61. The method of Clause 60, wherein the second elongate structure is the elongate structure of any one of the preceding Clauses.

Clause 62. The method of any one of the preceding Clauses, further comprising applying heat to the first portion, the second portion, or the first and second portions for a period of time at a predetermined temperature.

Clause 63. The method of Clause 62, wherein the period of time is at least 20 minutes, 25 minutes, or 30 minutes, 45 minutes, 60 minutes, 75 minutes, 90 minutes, or 120 minutes.

Clause 64. The method of Clause 62 or Clause 63, wherein the predetermined temperature is at least 300° C., 325° C., 350° C., 375° C., 400° C., 425° C., or 450° C.

Clause 65. The method of any one of Clause 62-Clause 64, wherein applying heat occurs before removing portions of the first elongate structure.

Clause 66. The method of any one of Clause 62-Clause 64, wherein applying heat occurs after removing portions of the first elongate structure.

Clause 67. The method of any one of Clause 62-Clause 66, wherein applying heat comprises applying heat only to discrete portion of the first portion, the second portion, or the first and second portions.

Clause 68. The method of any one of Clause 62-Clause 67, wherein applying heat causes the corresponding heat-treated area of the first or second elongated structure to have a different flexibility, stiffness, and/or pushability than a non-heat-treated area of the first or second elongated structure.

Clause 69. The method of any one of Clause 62-Clause 68, wherein applying heat increases the modulus of the corresponding heat-treated area of the first or second elongated structure relative to a non-heat-treated area of the first or second elongated structure.

Clause 70. The method of any one of Clause 62-Clause 69, wherein applying heat comprises applying heat via an inductive heater.

Clause 71. The method of any one of the preceding Clauses, wherein after removing portions of the first elongate structure, a distalmost section of the second portion is substantially straight.

Clause 72. The core member of any one of the preceding Clauses, wherein after removing portions of the first elongate structure, a distalmost section of the second portion does not exhibit curling or pig tailing.

Clause 73. The method of any one of the preceding Clauses, wherein the minimum length is at least 0.5 inches, 1.0 inches, 1.5 inches, 2.0 inches, 2.5 inches, 3.0 inches, 3.5 inches, 4.0 inches, 4.5 inches, 5.0 inches, 5.5 inches, or 6.0 inches.

Clause 74. The method of any one of the preceding Clauses, wherein removing comprises grinding the first elongate structure such that at least one of the first or second portions is continuously tapered along its length in the distal direction.

Clause 75. The method of any one of the preceding Clauses, wherein removing comprises grinding the second portion such that the outermost cross-sectional of the second portion is at least 0.0050 inches, 0.0055 inches, 0.0060 inches, 0.0065 inches, 0.0070 inches, 0.0075 inches.

Clause 76. The method of any one of the preceding Clauses, wherein removing comprises grinding the second portion such that the distal end outermost cross-sectional of the second portion is no more than 0.0015 inches, 0.0020 inches, 0.0025 inches, 0.0030 inches, 0.0035 inches, 0.0040 inches, 0.0045 inches, 0.0050 inches, 0.0055 inches, 0.0060 inches, 0.0065 inches, or 0.0070 inches.

Clause 77. The method of any one of the preceding Clauses, wherein removing comprises grinding the second portion such that the proximal end of the outermost cross-sectional of the second portion is no more than 0.0025 inches, 0.0030 inches, 0.0035 inches, 0.0040 inches, 0.0045 inches, 0.0050 inches, 0.0055 inches, 0.0060 inches, 0.0065 inches, 0.0070 inches, 0.0075 inches, 0.0080 inches, or 0.0085 inches.

Clause 78. The method of any one of the preceding Clauses, wherein removing comprises grinding the first elongate structure such that the second elongate structure comprises a ground portion and an unground portion.

Clause 79. A method of operating a medical device delivery system, the method comprising:
inserting the core member of any one of the preceding Clauses into a lumen of a catheter, wherein a stent or implant extends longitudinally over at least a portion of the core member; and
advancing the core member through the catheter.

Clause 80. The method of any one of the preceding Clauses, wherein advancing the core member comprises distally advancing the core member such that at least a portion of the stent or implant is allowed to extend out of the core member and expand.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is a cross-sectional side view of a medical device delivery system, in accordance with embodiments of the present technology.

FIG. 2A is a cross-sectional side view of a core member, in accordance with embodiments of the present technology.

FIGS. 2B-2D are cross-sectional end views of different portions of the core member shown in FIG. 2A.

FIGS. 3 and 4 are flow diagrams illustrating methods for manufacturing a core member, in accordance with embodiments of the present technology.

FIGS. 5A-5C are cross-sectional side views of portions of the core member shown in FIG. 2A at different stages during of the method illustrated in FIG. 4, in accordance with embodiments of the present technology.

FIGS. 6A and 6B are images of respective treated and untreated core members, in accordance with embodiments of the present technology.

### DETAILED DESCRIPTION

Conventional medical device delivery systems include an elongate core member (e.g., a guidewire or pushwire), which is used to carry and/or deliver a medical device (e.g., a stent, implant, therapeutic instrument, retrieval device, stent retriever, coil, filter, valve, prosthesis) to a target treatment site of a patient. To navigate through a patient's tortuous anatomy, a core member can be manufactured to have particular characteristics (e.g., stiffness, flexibility, and/or pushability) that vary along its length. For example, a proximal section of the core member may have a higher stiffness than that of a distal section of the core member to enhance pushability at the proximal section and/or to enhance flexibility at the distal section. To provide such characteristics, the core member, when implemented as a wire, may be made from multiple materials in a concentrically layered or nested fashion, with a first inner material surrounded by a cylindrical shell or layer of a second outer material.

Despite this intended effect, however, core members are susceptible to limitations that can reduce some of their functionality. For example, to provide a particular flexibility at their distalmost ends, core members implemented in the form of a wire are often tapered in a distal direction such that their distalmost ends have a diameter that can be less than approximately 0.050 inches. During manufacturing, when radially outer portions of a core member in the form of a wire having an inner, relatively flexible material surrounded by an outer, relatively stiff material are removed (e.g., by grinding), thus reducing the distal portion of the core member to the flexible inner material and little or none of the stiff outer material, such removal causes the distalmost end and adjacent portions of the core member to exhibit curling, “pigtailing,” or a general waviness, all of which are generally undesired for use in medical device delivery systems. Additionally, forming the core members from multiple materials drawn or fused together, which may be done to improve strength characteristics, can have its own disadvantages. For example, the multiple materials, compared to a single material, can inhibit translation of the pushability and/or flexibility characteristics along the length of the core members, including at the distalmost end. As a result, the intended use of core members for medical device delivery systems is limited.

Embodiments of the present technology provide an improved core member that reduces the risk of such issues. For example, embodiments of the present technology can comprise a core material that extends substantially along the length of the core member, and a shell material that surrounds the core material for at least a portion of the length. The core member can be tapered in a distal direction such that the distalmost portion of the core member includes only the core material, or the core material surrounded by only a relatively thin or relatively thinnest shell of the shell material. As explained in detail elsewhere herein, in some embodiments portions of the core member may be treated (e.g., heat treated) and/or aged, e.g., to increase corresponding strength modulus of those treated portions and/or enable those treated portions to have desirable pushability and/or flexibility characteristics. As a result of heat treating, corresponding portions of the core member may not exhibit the curling, pigtailing, or general waviness after grinding or other methods of tapering the core member occur. Instead, the treated portions of the core member may have a sufficiently high strength modulus to withstand these undesirable features and maintain a substantially straight profile that does not substantially impede the intended use thereof.

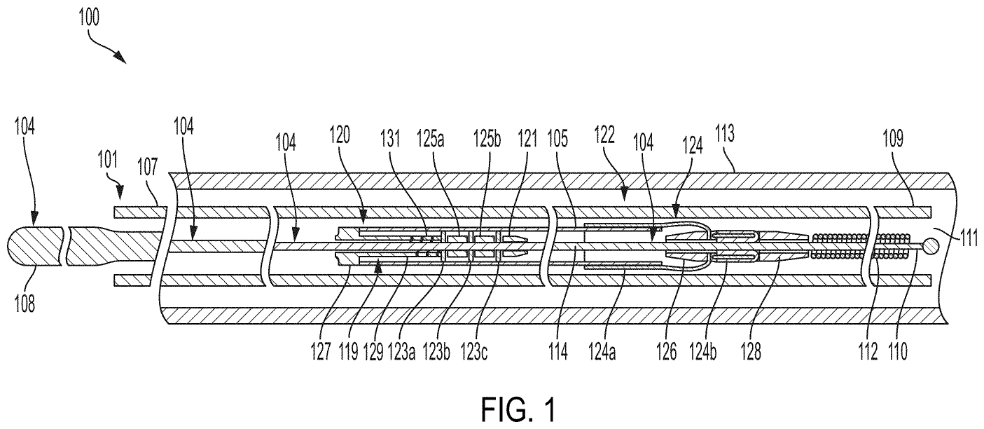

Specific details of several embodiments of the present technology are described herein with reference to FIGS. 1-6A. Although many of the embodiments are described with respect to devices, systems, and methods for delivery of stents and other medical devices, other applications and other embodiments in addition to those described herein are within the scope of the present technology. Further, embodiments of the present technology can have different configurations, components, and/or procedures than those shown or described herein. Moreover, embodiments of the present technology can have configurations, components, and/or procedures in addition to those shown or described herein and these and other embodiments may not have several of the configurations, components, and/or procedures shown or described herein without deviating from the present technology.

As used herein, the terms “distal” and “proximal” define a position or direction with respect to a clinician or a clinician's control device (e.g., a handle of a delivery catheter). For example, the terms, “distal” and “distally” refer to a position distant from or in a direction away from a clinician or a clinician's control device along the length of device. In a related example, the terms “proximal” and “proximally” refer to a position near or in a direction toward a clinician or a clinician's control device along the length of device. The headings provided herein are for convenience only and should not be construed as limiting the subject matter disclosed.

FIGS. 1-6A depict embodiments of medical device delivery systems or portions thereof that may be used to deliver and/or deploy a medical device into a hollow anatomical structure such as a blood vessel. FIG. 1 is a side cross-sectional view of a medical device delivery system 100 configured in accordance with embodiments of the present technology. The delivery system 100 can be configured to carry a stent (or other vascular implant or device) 105 thereon to be advanced through a surrounding elongate tube or catheter 101 to a target site in a patient, for example, a site within a corporeal lumen 113 such as a blood vessel. The catheter 101 can slidably receive a core member 104 configured to carry the stent 105 thereon. The depicted catheter 101 has a proximal portion 107 and an opposing distal portion 109, which can be positioned at a treatment site within a patient, and an internal lumen 111 extending from the proximal portion 107 to the distal portion 109. At the distal portion 109, the catheter 101 has a distal opening through which the core member 104 may be advanced beyond the distal portion 109 to expand or deploy the stent 105 within the corporeal lumen 113. The proximal portion 107 may include a catheter hub (not shown). The catheter 101 can define a generally longitudinal dimension extending between the proximal portion 107 and the distal portion 109. When the delivery system 100 is in use, the longitudinal dimension need not be straight along some or any of its length.

The delivery system 100 can be used with any number of catheters. For example, the catheter 101 can optionally comprise any of the various lengths of the MARKSMAN™ catheter available from Medtronic Neurovascular of Irvine, Calif. USA. The catheter 101 can optionally comprise a microcatheter having an inner diameter of about 0.030 inches or less, and/or an outer diameter of 3 French or less near the distal portion 109. Instead of or in addition to these specifications, the catheter 101 can comprise a microcatheter which is configured to percutaneously access the internal carotid artery, or another location within the neurovasculature distal of the internal carotid artery.

The core member 104 can generally comprise any member(s) with sufficient flexibility and column strength to move the stent 105 or other medical device through the catheter 101. For example, the core member 104 can comprise a wire (e.g., drawn filled tube (DFT) wire). Additionally or alternatively, in some embodiments the core member 104 can include a tube (e.g., hypotube), braid, coil, or other suitable member(s), or a combination of wire(s), tube(s), braid(s), coil(s), etc.

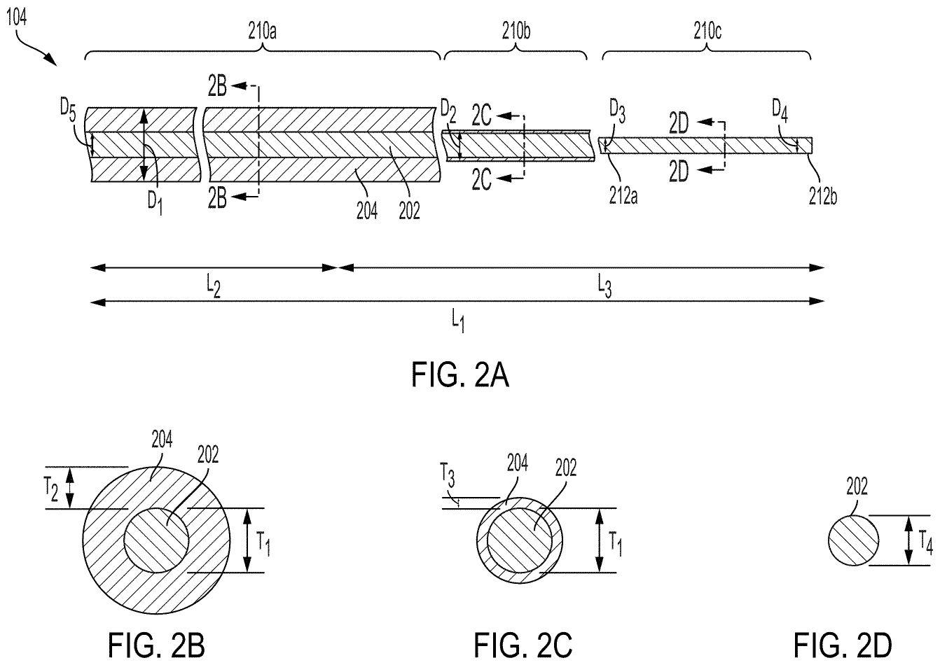

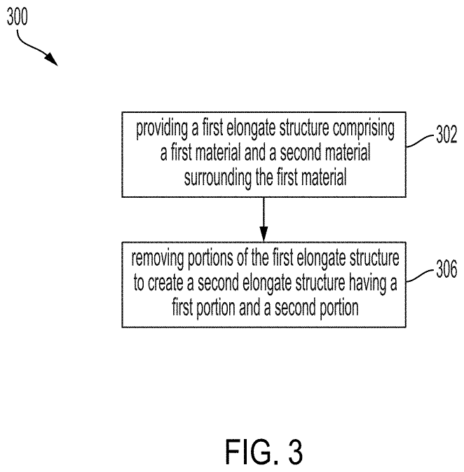

The core member 104 includes a proximal portion 108 and a distal portion 110, which can optionally include a tip coil 112. The core member 104 can be constructed from materials including polymers and metals including nitinol and stainless steels. In some embodiments, the core member 104 tapers in the distal direction, having a larger diameter at the proximal portion 108 and a smaller diameter at the distal portion 110. The taper may be gradual and continuous along the length of the core member 104, or in some embodiments the taper may be intermittent such that the core member 104 includes one or more tapering sections, and one or more constant diameter sections extending between (and/or adjacent to) the tapering section(s). Some embodiments described in more detail elsewhere herein (e.g., with reference to FIGS. 2A-2D) include an overall tapering core member 104 which can include one or more such constant-diameter segments in which the core member 104 does not taper. Such constant-diameter segments can be useful, e.g., for utilizing a single wire in combination with stents 105 of different lengths.

The core member 104 can also include an intermediate portion 114 located between the proximal portion 108 and the distal portion 110. The intermediate portion 114 includes the portion of the core member 104 onto or over which the stent 105 extends when the delivery system 100 is in the pre-deployment configuration as shown in FIG. 1. The core member 104 may include one or more fluorosafe markers (not shown) disposed at one or more locations along the length of the core member 104.

The system 100 can also include a coupling assembly 120 or resheathing assembly 120 configured to releasably retain the medical device or stent 105 with respect to the core member 104. The coupling assembly 120 can be configured to engage the stent 105, e.g., via mechanical interlock with the pores and filaments of the stent 105, abutment of the proximal end or edge of the stent 105, frictional engagement with the inner wall of the stent 105, or any combination of these modes of action. The coupling assembly 120 can therefore cooperate with the overlying inner surface of the catheter 101 to grip and/or abut the stent 105 such that the coupling assembly 120 can move the stent 105 along and within the catheter 101. For example, distal and/or proximal movement of the core member 104 relative to the catheter 101 can result in a corresponding distal and/or proximal movement of the stent 105 within the catheter lumen 111.

The coupling assembly 120 (or portion(s) thereof) can, in some embodiments, be configured to rotate about the core member 104. In some such embodiments, the coupling assembly 120 can comprise a proximal restraint 119 and a distal restraint 121. The proximal and distal restraints 119, 121 can be fixed to the core member 104 to prevent or limit proximal or distal movement of the coupling assembly 120 along the longitudinal dimension of the core member 104. For example, one or both of the proximal and distal restraints 119, 121 can be soldered or fixed, e.g., with adhesive, to the core member 104. One or both of the proximal and distal restraints 119, 121 can have an outside diameter or other radially outermost dimension that is smaller than the outside diameter or other radially outermost dimension of the overall coupling assembly 120 such that an outer profile of one or both of the restraints 119, 121 is positioned radially inward of the inner surface of the stent 105 during operation of the system 100. In some embodiments, the proximal restraint 119 can be sized to abut the proximal end of the stent 105, e.g., to prevent or inhibit the stent from traveling distally thereof during delivery. The proximal and distal restraints 119, 121 can comprise a metal such as platinum, iridium, gold, tungsten or combinations thereof (e.g., 90% platinum/10% iridium).

The proximal restraint 119 can include a bumper section 127 and a distal section 129 that extends distally from the bumper section 127. The distal section 129 can be a tubular member having a helical cut extending along at least a portion of its length, thereby forming a proximal spiral-cut section 131. In some embodiments, the proximal spiral-cut section 131 and the proximal restraint 119 can be formed as an integrally formed, single component such that the proximal spiral-cut section 131 comprises part of the proximal restraint 119. In other embodiments, the proximal spiral-cut section 131 can be formed individually, separate from the proximal restraint 119 and coupled thereto. In operation, the stent 105 may extend over the distal section 129 of the proximal restraint 119, such that a proximal end of the stent 105 abuts the bumper section 127 of the proximal restraint 119. As the coupling assembly 120 is distally advanced through the lumen 111 of the catheter 101 (or as the catheter 101 is proximally retracted relative to the coupling assembly 120), the bumper section 127 of the proximal restraint 119 can “push” the stent 105 or otherwise inhibit or prevent relative proximal movement of the stent 105 proximally beyond the bumper section 127 of the proximal restraint 119.

The coupling assembly 120 can also include one, two, three or more stent engagement members (or device engagement members, or resheathing members) and one, two or more spacers disposed about the core member 104 between the proximal and distal restraints 119, 121. In the illustrated embodiment, the coupling assembly 120 includes first, second and third stent engagement members 123a-c (collectively referred to as “engagement members 123”) and first and second spacers 125a-b (collectively referred to as “spacers 125”) disposed over the core member 104. In a distal direction, the elements of the coupling assembly 120 include the proximal restraint 119, the first stent engagement member 123a, the first spacer 125a, the second stent engagement member 123b, the second spacer 125b, the third stent engagement member 123c and the distal restraint 121. The first spacer 125a defines the relative longitudinal spacing between the first engagement member 123a and the second engagement member 123b, and the second spacer 125b defines the relative longitudinal spacing between the second engagement member 123b and the third engagement member 123c.

The stent engagement members 123 and the spacers 125 (or any of the engagement members or spacers disclosed herein) can be fixed to the core member 104 so as to be immovable relative to the core member 104, in a longitudinal/sliding manner and/or in a radial/rotational manner. Alternatively, the spacers 125 and/or the stent engagement members 123 can be coupled to (e.g., mounted on) the core member 104 so that the spacers 125 and/or the stent engagement members 123 can rotate about the longitudinal axis of the core member 104, and/or move or slide longitudinally along the core member 104. In such embodiments, the spacers 125 and/or the stent engagement members 123 can each have an inner lumen or aperture that receives the core member 104 therein such that the spacers 125 and/or the stent engagement members 123 can slide and/or rotate relative to the core member 104.

In some embodiments, the proximal and distal restraints 119, 121 can be spaced apart along the core member 104 by a longitudinal distance that is slightly greater than the combined length of the spacers 125 and the stent engagement members 123, so as to leave one or more longitudinal gaps between the individual spacers 125, engagement members 123, and/or proximal and distal restraints 119, 121, When present, the longitudinal gap(s) allow the spacers 125 and/or the stent engagement members 123 to slide longitudinally along the core member 104 between the restraints 119, 121. The longitudinal range of motion of the spacers 125 and the stent engagement members 123 between the restraints 119, 121 is approximately equal to the total combined length of the longitudinal gap(s), if any. In some embodiments, the combined length of the longitudinal gap(s) between the proximal restraint 119 and the distal restraint 121 can be 0.05 mm or less. In various embodiments, such longitudinal gap(s) can facilitate rotatability of the engagement members 123 and/or spacers 125 about the core member 104. Such longitudinal gaps(s) can also improve bendability of the core member 104 and the distal restraint 121 about a relatively sharp radius of curvature, as may be required when navigating the tortuous anatomy of a patient's neurovasculature.

One or both of the spacers 125 can take the form of a wire coil, a solid tube, or other structural element that can be mounted over the core member 104 to longitudinally separate adjacent components of the coupling assembly 120. In some embodiments, one or both of the spacers 125 can be a zero-pitch coil with flattened ends. In some embodiments, one or both of the spacers 125 can be a solid tube (e.g., a laser-cut tube) that can be rotatably mounted or non-rotatably fixed (e.g., soldered) to the core member 104. The spacers 125 can have a radially outermost dimension that is smaller than a radially outermost dimension of adjacent components, e.g., the engagement members 123 such that the spacers 125 do not contact the stent 105 during normal operation of the system 100. The dimensions, construction, and configuration of the spacers 125 can be selected to achieve improved grip between the coupling assembly 120 and the overlying stent 105.

The stent 105 can comprise a braided stent or other form of stent such as a woven stent, knit stent, laser-cut stent, roll-up stent, etc. The stent 105 can optionally be configured to act as a “flow diverter” device for treatment of aneurysms, such as those found in blood vessels including arteries in the brain or within the cranium, or in other locations in the body such as peripheral arteries. The stent 105 can optionally be similar to any of the versions or sizes of the PIPELINE™ Embolization Device marketed by Medtronic Neurovascular of Irvine, Calif. USA. The stent 105 can alternatively comprise any suitable tubular medical device and/or other features as described herein. In some embodiments, the stent 105 can be any one of the stents described in U.S. application Ser. No. 15/892,268, filed Feb. 8, 2018, titled VASCULAR EXPANDABLE DEVICES, the entirety of which is hereby incorporated by reference herein and made a part of this specification.

The stent 105 can be moved distally or proximally within the overlying catheter 101 via the proximal coupling assembly 120. In some embodiments, the stent 105 can be resheathed via the proximal coupling assembly 120 after partial deployment of the stent 105 from a distal opening of the catheter. In embodiments in which the proximal restraint 119 is sized to abut the proximal end of the stent 105 and employed to push the stent distally during delivery, the first and second stent engagement members 123a-b can be employed to resheath the stent 105 after partial deployment, while taking no (or substantially no) part in pushing the stent distally during delivery. For example, the first and second stent engagement members 123a-b can in such embodiments transmit no, or substantially no, distal push force to the stent 105 during delivery.

Optionally, the proximal edge of the proximal coupling assembly 120 can be positioned just distal of the proximal edge of the stent 105 when in the delivery configuration. In some such embodiments, this enables the stent 105 to be re-sheathed when as little as a few millimeters of the stent remains in the catheter. Therefore, with stents of typical length, resheathability of 75% or more can be provided (i.e., the stent can be re-sheathed when 75% or more of it has been deployed).

With continued reference to FIG. 1, the distal interface assembly 122 can comprise a distal engagement member 124 that can take the form of, e.g., a distal device cover or distal stent cover (generically, a “distal cover”). The distal cover 124 can be configured to reduce friction between the stent 105 (e.g., a distal portion thereof) and the inner surface of the surrounding catheter 101. For example, the distal cover 124 can be configured as a lubricious, flexible structure having a free first end or section 124a that can extend over at least a portion of the stent 105 and/or intermediate portion 108 of the core member 104, and a fixed second end or section 124b that can be coupled (directly or indirectly) to the core member 104.

The distal cover 124 can have a first or delivery position, configuration, or orientation in which the distal cover can extend proximally relative to the distal tip, or proximally from the second section 124b or its (direct or indirect) attachment to the core member 104, and at least partially surround or cover a distal portion of the stent 105. The distal cover 124 can be movable from the first or delivery orientation to a second or resheathing position, configuration, or orientation (not shown) in which the distal cover can be everted such that the first end 124a of the distal cover is positioned distally relative to the second end 124b of the distal cover 124 to enable the resheathing of the core member 104, either with the stent 105 carried thereby, or without the stent 105. As shown in FIG. 1, the first section 124a of the distal cover 124 can originate from the proximal end of the second section 124b. In another embodiment, the first section 124a can originate from the distal end of the second section 124b.

The distal cover 124 can be manufactured to include a lubricious and/or hydrophilic material such as PTFE or Teflon®, but may be made from other suitable lubricious materials or lubricious polymers. The distal cover can also comprise a radiopaque material which can be blended into the main material (e.g., PTFE) to impart radiopacity. The distal cover 124 can have a thickness of between about 0.0005″ and about 0.003″. In some embodiments, the distal cover can be one or more strips of PTFE having a thickness of about 0.001″.

The distal cover 124 (e.g., the second end 124b thereof) can be fixed to the core member 104 (e.g., to the core member 104 or distal tip thereof) so as to be immovable relative to the core member 104, either in a longitudinal/sliding manner or a radial/rotational manner. Alternatively, as depicted in FIG. 1, the distal cover 124 (e.g., the second end 124b thereof) can be coupled to (e.g., mounted on) the core member 104 so that the distal cover 124 can rotate about a longitudinal axis of the core member 104 (e.g., of the core member 104), and/or move or slide longitudinally along the core member 104. In such embodiments, the second end 124b can have an inner lumen that receives the core member 104 therein such that the distal cover 124 can slide and/or rotate relative to the core member 104. Additionally, in such embodiments, the distal interface assembly 122 can further comprise a proximal restraint 126 that is fixed to the core member 104 and located proximal of the (second end 124b of the) distal cover 124, and/or a distal restraint 128 that is fixed to the core member 104 and located distal of the (second end 124b of the) distal cover 124. The distal interface assembly 122 can comprise a radial gap between the outer surface of the core member 104 (e.g., of the core member 104) and the inner surface of the second end 124b. Such a radial gap can be formed when the second end 124b is constructed with an inner luminal diameter that is somewhat larger than the outer diameter of the corresponding portion of the core member 104. When present, the radial gap allows the distal cover 124 and/or second end 124b to rotate about the longitudinal axis of the core member 104 between the proximal and distal restraints 126, 128.

In some embodiments, one or both of the proximal and distal restraints 126, 128 of the distal interface assembly 122 can have an outside diameter or other radially outermost dimension that is smaller than the (e.g., pre-deployment) outside diameter or other radially outermost dimension of the distal cover 124, so that one or both of the restraints 126, 128 will tend not to bear against or contact the inner surface of the catheter during operation of the core member 104. Alternatively, it can be preferable to make the outer diameters of the restraints 126 and 128 larger than the largest cross-sectional of the pre-deployment distal cover 124, and/or make the outer diameter of the proximal restraint 126 larger than the outer diameter of the distal restraint 128. This configuration allows easy and smooth retrieval of the distal cover 124 and the restraints 126, 128 back into the catheter post stent deployment.

In operation, the distal cover 124, and in particular the first section 124a, can generally cover and protect a distal portion of the stent 105 as the stent 105 is moved distally through a surrounding catheter. The distal cover 124 may serve as a bearing or buffer layer that, for example, inhibits filament ends of the distal portion of the stent 105 (where the stent comprises a braided stent) from contacting an inner surface of the catheter, which could damage the stent 105 and/or catheter, or otherwise compromise the structural integrity of the stent 105. Since the distal cover 124 may be made of a lubricious material, the distal cover 124 may exhibit a low coefficient of friction that allows the distal portion of the stent to slide axially within the catheter with relative ease. The coefficient of friction between the distal cover 124 and the inner surface of the catheter 101 can be between about 0.02 and about 0.4. For example, in embodiments in which the distal cover and the catheter are formed from PTFE, the coefficient of friction can be about 0.04. Such embodiments can advantageously improve the ability of the core member 104 to pass through the catheter, especially in tortuous vasculature.

Structures other than those embodiments of the distal cover 124 described herein may be used in the core member 104 and/or distal interface assembly 122 to cover or otherwise interface with the distal portion of the stent 105. For example, a protective coil or other sleeve having a longitudinally oriented, proximally open lumen may be employed. In other embodiments, the distal interface assembly 122 can omit the distal cover 124, or the distal cover can be replaced with a component similar to the proximal coupling assembly 120. Where the distal cover 124 is employed, it can be connected to the distal tip coil 112 (e.g., by being wrapped around and enclosing some or all of the winds of the coil 112) or being adhered to or coupled to the outer surface of the coil by an adhesive or a surrounding shrink tube. The distal cover 124 can be coupled (directly or indirectly) to other portions of the core member 104, such as the core member 104.

In embodiments of the core member 104 that employ both a rotatable proximal coupling assembly 120 and a rotatable distal cover 124, the stent 105 can be rotatable with respect to the core member 104 about the longitudinal axis thereof, by virtue of the rotatable connections of the proximal coupling assembly 120 and distal cover 124. In such embodiments, the stent 105, proximal coupling assembly 120, and distal cover 124 can rotate together in this manner about the core member 104. When the stent 105 can rotate about the core member 104, the core member 104 can be advanced more easily through tortuous vessels as the tendency of the vessels to twist the stent 105 and/or core member 104 is negated by the rotation of the stent 105, proximal coupling assembly 120, and distal cover 124 about the core member 104. In addition, the required push force or delivery force is reduced, as the user's input push force is not diverted into torsion of the stent 105 and/or core member 104. The tendency of a twisted stent 105 and/or core member 104 to untwist suddenly or “whip” upon exiting tortuosity or deployment of the stent 105, and the tendency of a twisted stent to resist expansion upon deployment, are also reduced or eliminated. Further, in some such embodiments of the core member 104, the user can “steer” the core member 104 via the tip coil 112, particularly if the coil 112 is bent at an angle in its unstressed configuration. Such a coil tip can be rotated about a longitudinal axis of the system 100 relative to the stent 105, coupling assembly 120 and/or distal cover 124 by rotating the distal portion 110 of the core member 104. Thus the user can point the coil tip 112 in the desired direction of travel of the core member 104, and upon advancement of the core member the tip will guide the core member in the chosen direction.

FIG. 2A is a cross-sectional side view of a core member 104 which can, in some embodiments, be similar to that shown and described with reference to FIG. 1, and incorporate additional features as further described below. FIGS. 2B-2D are cross-sectional end views of portions of the core member 104. As shown in FIG. 2A, the core member 104 can include a first portion 210a, a second portion 210b distal to the first portion 210a, and a third portion 210c distal to the second portion 210b. The core member 104 includes a first material (e.g., a core material) 202 extending along substantially an entire length (L₁) of the core member 104, and a second material 204 extending along the first and second portions 210a-b or otherwise along only a portion of the length (L₁) (or along the entire length L₁). The second material 204 surrounds or at least partially surrounds the first material 202 in the first and second portions 210a-b, and may be fused, soldered, or otherwise fixedly secured to the first material 202, e.g. via the process of forming or drawing the materials 202, 204 into a wire. As shown in FIG. 2A, the first and second portions 210a-b may include the first material 202 and the second material 204, and the third portion 210c may include only the first material 202.

The first and second materials 202, 204 can comprise different materials; e.g., the first material can comprise a first metal or alloy and the second material can comprise a second metal alloy different in material composition and/or mechanical properties from the first metal or alloy. The first and second materials can each comprise a metal and/or polymer material. For example, the first and/or second materials 202, 204 can each comprise nitinol, titanium (e.g., titanium beta III), 35N LT (e.g., the 35N LT high performance alloy marketed by Fort Wayne Metals Research Products of Fort Wayne, Ind. USA), stainless steel, chromium, cobalt, and/or alloys thereof (e.g., cobalt-chromium (“Co—Cr”). The core member 104 may comprise any combinations of these materials. For example, in some embodiments the first material 202 can comprise a Co—Cr alloy and the second material 204 can comprise titanium, e.g. titanium beta III. Alternatively, the first material 202 can comprise 35N LT and the second material 204 can comprise titanium beta III. In some embodiments, the first material can have a lower modulus of elasticity and/or a higher stiffness than the second material.

The core member 104 can taper in the distal direction, such that the maximum outermost cross-sectional dimension (e.g., diameter) (D₁) of the first portion 210a is larger than the maximum outermost cross-sectional dimension (D₂) of the second portion 210b, which is larger than the maximum outermost cross-sectional dimension (D₃) of the third portion 210c. The outermost cross-sectional dimension (D₁) of the first portion 210a or second material 204 can be at least 0.010 inches, or at least 0.015 inches (e.g., about 0.018 inches). In addition to or in lieu of the foregoing, the outermost cross-sectional dimension (D₅) of the first material 202 of the first portion 210a can be at least 0.0050 inches (e.g., about 0.0070 inches). The taper may be gradual and continuous along the length (L₁) of the core member 104, or in some embodiments the taper may vary at different portions of the core member 104, or the taper can be intermittent, with tapering sections separated by one or more constant-diameter sections extending between or adjacent to the tapering section(s). As a result of the tapering, with reference to FIGS. 2B and 2C, the thickness (T₂) of the second material 204 in the first portion 210a is greater than the thickness (T₃) of the second material 204 in the second portion 210b. Additionally, with reference to the FIGS. 2C and 2D, the thickness (T₁) of the first material 202 in the first and second portions 210a-b can be the same, and can be greater than the thickness (T₄) of the first material 202 in the third portion 210c.

The core member 104 may be tapered via grinding or other known methods in the art. In embodiments where grinding is used to remove a portion of the outermost material of the core member 104, the core member 104 may have an unground length (L₂) in which no material has been removed, and a ground length (L₃) in which a portion of the first and/or second materials have been removed. The unground length (L₂) can be at least 30 inches, 35 inches, 40 inches, 45 inches, or 50 inches. Additionally or alternatively, the ground length (L₃) can be at least 20 inches, 25 inches, 30 inches, 35 inches, 40 inches, 45 inches, or 50 inches. The ground length (L₃) for a particular core member 104 may be determined based on a desired pushability, stiffness, and/or flexibility of the different portions of the corresponding core member 104, and/or the distance from the contemplated access site to the contemplated treatment site.

The third portion 210c of the core member 104 can include an outermost cross-sectional dimension that tapers in the distal direction. In some embodiments, the outermost cross-sectional dimension (D₃) of the third portion 210c at its proximal end 212a is no more than 0.0025 inches, 0.0030 inches, 0.0035 inches, 0.0040 inches, 0.0045 inches, 0.0050 inches, 0.0055 inches, 0.0060 inches, 0.0065 inches, 0.0070 inches, 0.0075 inches, 0.0080 inches, or 0.0085 inches. In addition to or in lieu of the foregoing, the outermost cross-sectional dimension (D₄) of the third portion 210c at its distal end 212b is less than the outermost cross-sectional dimension (D₃) and in some embodiments is no more than 0.0025 inches, 0.0030 inches, 0.0035 inches, 0.0040 inches, 0.0045 inches, 0.0050 inches, 0.0055 inches, 0.0060 inches, 0.0065 inches, 0.0070 inches, 0.0075 inches, 0.0080 inches, or 0.0085 inches. In some embodiments, the third portion 210c can comprise a minimum length of at least 0.5 inches, 1.0 inches, 1.5 inches, 2.0 inches, 2.5 inches, 3.0 inches, 3.5 inches, 4.0 inches, 4.5 inches, 5.0 inches, 5.5 inches, or 6.0 inches. The minimum length of the third portion 210c (and/or the second portion 210b) may be substantially straight when unstressed, and not exhibit curling, waviness, or pigtailing. As explained in additional detail elsewhere herein, the third portion 210c (and/or the second portion 210b) or discrete areas thereof may be treated to increase corresponding strength moduli.

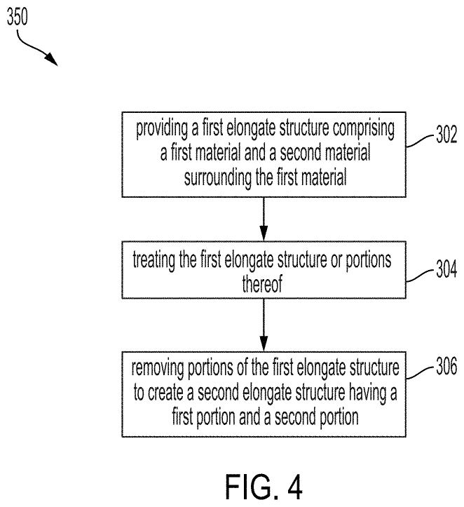

FIG. 3 is a flow diagram illustrating a method 300 for manufacturing a core member (e.g., the core member 104; FIGS. 1 and 2A), in accordance with embodiments of the present technology. The method 300 includes providing a first elongate structure comprising a first material (e.g., the first material 202; FIG. 2A) and a second material (e.g., the second material 204; FIG. 2A) at least partially surrounding the first material along at least a portion of the length of the first elongate structure (process portion 302). The method 300 further comprises removing portions of the first elongate structure to form a second elongate structure comprising (i) a first portion (e.g., the first portion 210a; FIG. 2A) including the first and second materials, and (ii) a second portion (e.g., the third portion 210c; FIG. 2A) distal to the first portion and including only the first material (process portion 306). Removing portions of the first elongate structure can include grinding an outer surface of the first elongate structure to form the tapered second elongate structure.

FIG. 4 is a flow diagram illustrating a method 350 for manufacturing a core member (e.g., the core member 104; FIGS. 1 and 2A), in accordance with embodiments of the present technology. The method 350 includes process portions 302 and 306 previously described, and additionally includes treating the first elongate structure (e.g., the first and/or second materials) or portions thereof (process portion 304). As described elsewhere herein, treating can include increasing a strength modulus of the treated portions of the first elongate structure. Process portion 304 can be performed before and/or after process portion 306. As explained elsewhere herein, in some embodiments treating the first elongate structure prior to removing portions thereof can inhibit or prevent the treated portions from curling or exhibiting pigtailing features after the portions of the first elongate structure are removed. In some embodiments, only a discrete portion (e.g., the third portion 210c (FIG. 2A) or a distal section of the third portion 210c) of the first elongate structure may be treated.

Treating can include applying heat to the first elongate structure at a predetermined temperature for a minimum period of time (often referred to as “aging”). Without being bound by theory, heat treating a material can affect its physical and chemical properties to thereby change its strength and softness/hardness characteristics. The predetermined temperature can be (i) at least 300° C., 325° C., 350° C., 375° C., 400° C., 425° C., or 450° C., or (ii) within a range of 300−450° C. or any incremental range therebetween (e.g., 330-380° C.), and the period of time can be (i) at least 20 minutes, 25 minutes, or 30 minutes, 45 minutes, 60 minutes, 75 minutes, 90 minutes, or 120 minutes, or (ii) within a range of 20-120 minutes or any incremental range therebetween. Treated portions of the first elongate structure may have a modulus of (i) at least 60 gigapascals (GPa), 65 GPa, 70 GPa, 75 GPa, 80 GPa, 85 GPa, 90 GPa, 95 GPa, or 100 GPa, or (ii) within a range of 60-100 GPa or any incremental range therebetween. In some embodiments, heat treating in such a manner can increase the modulus of the corresponding portion by at least 50%. Prior to heat treating, the modulus of the first elongate structure may be as low as approximately 40 GPa.

Treating the first elongate structure in the manner described herein can enable the first and/or second materials of the second elongate structure (e.g., the core member 104; FIGS. 1 and 2A) to remain substantially straight (e.g., when unstressed) at distal end portions thereof (e.g., the third portion 210c; FIG. 2A). That is, treating the first and/or second materials of a core member in the manner described herein can prevent the core member from exhibiting pigtailing, curling, or waviness, at least at the distal end portions, after grinding or other removal techniques have occurred. In doing so, the core member 104 is better able to carry out its intended use, such as carrying and/or delivering a medical device to a target site of a patient.

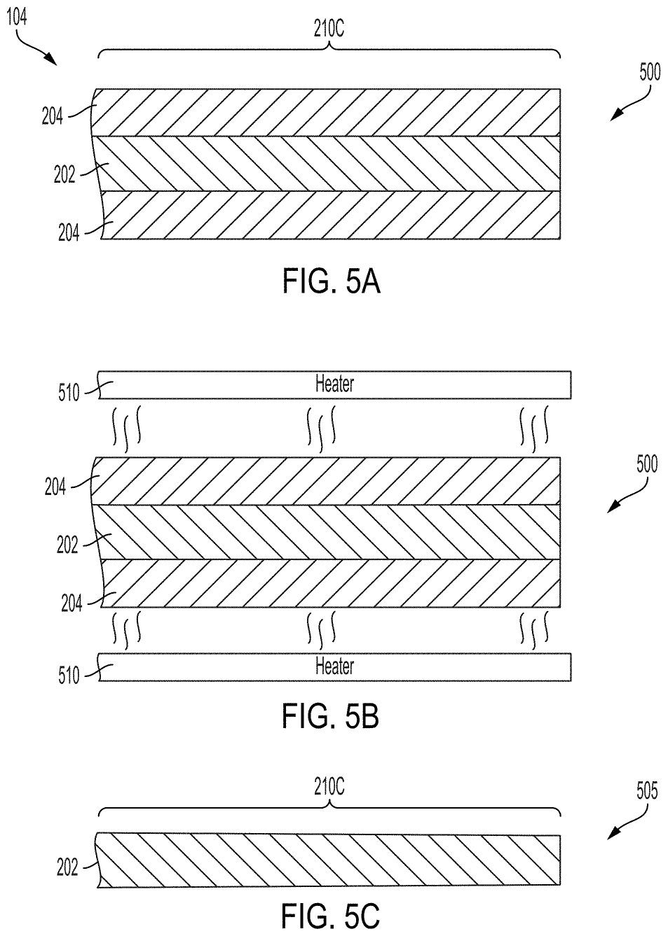

FIGS. 5A-5C illustrate cross-sectional side views of portions of the core member 104 shown in FIG. 2A at different stages during the method of FIG. 3 and/or FIG. 4. FIG. 5A illustrates a first elongate structure 500 corresponding to the third portion 210c (FIG. 2A) of the core member 104 before portions thereof are removed to form a tapered profile, as previously described. As such, the first elongate structure 500 includes the first material 202 surrounded by the second material 204.

FIG. 5B illustrates the first elongate structure 500 being heat treated via a heater 510 (e.g., a convection, inductive, or radiant heater). In some embodiments, the heater 510 can heat portions of the first elongated structure 500 (i.e., portions of the first and/or second materials 202, 204) to a predetermined temperature for a period of time. For example, the heater 510 may heat just a distal end portion of the first elongate structure 500 or discrete portions of the first elongate structure 500. That is, the heater 510 may be positioned to heat first and second areas of the first elongate structure 500, in which the first area is spaced apart from the second area. As mentioned previously, the predetermined temperature can be (i) at least 300° C., 325° C., 350° C., 375° C., 400° C., 425° C., or 450° C., or (ii) within a range of 300−450° C. or any incremental range therebetween, and the period of time can be (i) at least 20 minutes, 25 minutes, or 30 minutes, 45 minutes, 60 minutes, 75 minutes, 90 minutes, or 120 minutes, or (ii) within a range of 20-120 minutes or any incremental range therebetween. Heating the first and/or second materials in such a manner can affect their physical and chemical properties to thereby increase strength and alter softness/hardness characteristics. Treated portions of the first elongate structure may have a modulus of (i) at least 60 gigapascals (GPa), 65 GPa, 70 GPa, 75 GPa, 80 GPa, 85 GPa, 90 GPa, 95 GPa, or 100 GPa, or (ii) within a range of 60-100 GPa or any incremental range therebetween. The increase in modulus can correspond to an at least 50% improvement in strength relative to untreated portions.

FIG. 5C illustrates a second elongate structure 505 or the third portion 210c of the core member 104, which corresponds to the treated first elongated structure 500 after portions thereof have been removed, e.g., by grinding or other known techniques in the art. As shown in FIG. 5C, the second material 204 has been removed from the third portion 210c, which only includes a tapered first material 202. As explained in additional detail elsewhere herein, the third portion 210c, in part as a result of being heat treated, remains substantially straight (e.g., when unstressed) and does not exhibit curling, pigtailing, or general waviness.

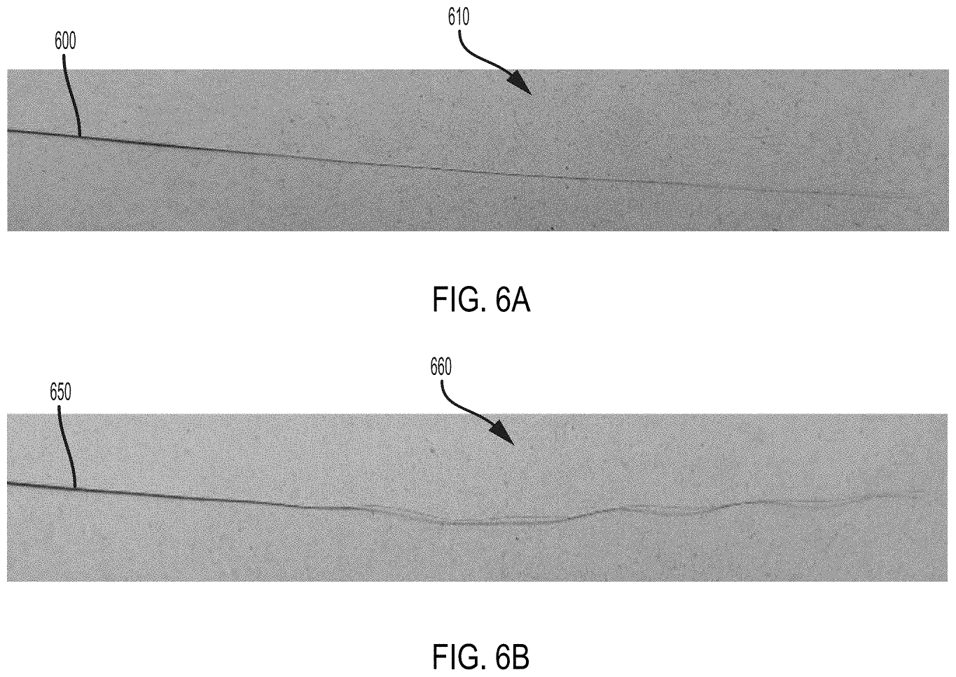

FIGS. 6A and 6B are images of respective treated and untreated grounded core members 600, 650, in accordance with embodiments of the present technology. Prior to grinding, the core members 600, 650 were 0.018 inch outer diameter wires made from a core material of titanium beta III and a shell material of 35N LT, which surrounds the core material. The wire 600 of FIG. 6A was heat treated before grinding, whereas the wire 650 of FIG. 6B was not heat treated before grinding. Specifically, prior to grinding, the distal end portion 610 of the wire 600 of FIG. 6A was exposed to 350° C. for approximately 120 minutes. As shown in FIG. 6A, the distal end portion 610 of the treated wire 600 after grinding remained substantially straight and did not exhibit curling or a wave-like shape. In contrast, as shown in FIG. 6B, the distal end portion 660 of the untreated wire 650 after grinding did exhibit curling and a general waviness (e.g., an oscillating shape). FIGS. 6A and 6B illustrate the effect that heat treating can have on core members, in accordance with embodiments of the present technology.

### CONCLUSION

Although many of the embodiments are described above with respect to systems, devices, and methods for manufacturing core members for use with medical devices, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-6A.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A stent delivery system, comprising:
a core member including—
a first portion comprising a first material and a second material surrounding the first material along a length of the first portion, the first material extending along substantially an entire length of the core member, and
a second portion distal to the first portion and comprising only the first material, the second portion being tapered in a distal direction such that an outermost cross-sectional dimension at a proximal end of the second portion is greater than an outermost cross-sectional dimension at a distal end of the second portion, the second portion having a minimum length of 1.0 inches; and
a stent carried by the core member.

2. The delivery system of claim 1, wherein the minimum length is at least 5 inches.

3. The delivery system of claim 1, wherein the outermost cross-sectional of the distal end of the second portion is no more than 0.0025 inches.

4. The delivery system of claim 1, wherein the outermost cross-sectional of the distal end of the second portion is no more than 0.006 inches.

5. The delivery system of claim 1, wherein the core member is ground such that the core member has a ground length of at least 20 inches and an unground length of at least 30 inches.

6. The delivery system of claim 1, wherein the second portion is heat-treated or aged.

7. The delivery system of claim 6, wherein a modulus of the core member measured at the distal end of the second portion is at least 80 GPa.

8. The delivery system of claim 1, wherein the second portion includes a first area having a first strength modulus and a second area having a second strength modulus greater than the first strength modulus, the second area being distal and tapered relative to the first area.

9. The delivery system of claim 1, wherein the first material comprises titanium beta III.

10. The delivery system of claim 9, wherein the second material comprises a cobalt-chromium alloy or 35N LT.

11. The delivery system of claim 1, wherein the second material in the first portion comprises a first thickness, the core member further comprising a third portion between the first and second portions, the third portion comprising the first material and the second material surrounding the first material, the second material in the third portion having a second thickness less than the first thickness.

12. A core member for use in delivering a medical device, comprising:
a first material extending along an entire length of the core member, the first material being tapered in a distal direction such that a thickness of the first material at a proximal end of the core member is greater than a thickness of the first material at a distal end of the core member,
a second material surrounding the first material for at least a portion of the length of the core member, the second material being tapered in the distal direction such that a thickness of the second material at the proximal end is greater than a thickness of the second material at the distal end, and
a distalmost section comprising (i) only the first material, (ii) an outermost cross-sectional dimension of no more than 0.070 inches, and (iii) a length of at least 0.5 inches, the distalmost section being substantially straight.

13. The core member of claim 12, wherein the distalmost section is heat-treated or aged.

14. The core member of claim 12, wherein a modulus of the distalmost section is at least 60 GPa.

15. The core member of claim 12, wherein the distalmost section is substantially straight when unstressed.

16. The core member of claim 12, wherein the first material comprises titanium beta III and the second material comprises a cobalt-chromium alloy or 35N LT.

17. The core member of claim 12, wherein the first material comprises platinum and the second material comprises 35N LT.

18. The core member of claim 12, wherein the first material comprises platinum and the second material comprises nitinol.

19. The core member of claim 12, wherein the first material comprises nitinol and the second material comprises 35N LT.

20. A method of manufacturing a core member for use in delivering a medical device, comprising:
providing a first elongate structure comprising a first material and a second material surrounding the first material, the first and second materials extending along a length of the first elongate structure; and
removing portions of the first elongate structure to form a second elongate structure, the second elongate structure comprising (i) a first portion including the first and second materials, and (ii) a second portion distal to the first portion and including only the first material,
wherein—
the first portion is tapered in a distal direction such that an outermost cross-sectional dimension at a proximal end of the first portion is greater than an outermost cross-sectional dimension at a distal end of the first portion,
the second portion is tapered in the distal direction such that an outermost cross-sectional dimension at a proximal end of the second portion is greater than an outermost cross-sectional dimension at a distal end of the second portion, and
the second portion has a length of at least 1.0 inches.

21. The method of claim 20, further comprising applying heat to the first portion, the second portion, or the first and second portions to increase a strength modulus thereof.

22. The method of claim 21, wherein applying heat comprising applying heat at a predetermined temperature of at least 350° C. for a period of time of at least 60 minutes.

23. The method of claim 21, wherein applying heat comprises applying heat to the first and second materials of the second portion, and wherein removing comprises removing portions of first elongate structure after applying heat.

24. The method of claim 23, wherein after removing portions of the first elongate structure, a distalmost section of the second portion does not exhibit curling or pig tailing.

25. The method of claim 23, wherein applying heat comprises applying heat such that a modulus of the heated second material of the second portion is at least 60 GPa.
